(19) United States
(12) Patent Application Publication
(10) Pub. No.: US 2017/0181934 A1
(43) Pub. Date: Jun. 29, 2017

(54) CLEANSING DEVICE HAVING ADDITIVE COMPOSITIONS DISPOSED IN A CARRIER

(71) Applicant: The Dial Corporation, Scottsdale, AZ (US)

(72) Inventors: Dan Khamis, Phoenix, AZ (US); Mary J. Conway, Phoenix, AZ (US)

(21) Appl. No.: 14/982,270

(22) Filed: Dec. 29, 2015

(51) Int. Cl. A61K 8/02; A61Q 19/10

(52) U.S. Cl. CPC A61K 8/0233; A61Q 19/10; A61K 2800/87; A61K 2800/88; A61K 2800/42

(57) ABSTRACT

A cleansing device is described. The cleansing device includes a carrier. The carrier has at least one cavity defined therein. The at least one cavity is to receive at least one additive composition and to maintain the at least one additive composition. The additive composition is to be applied to a surface. The device also includes a housing that holds the carrier and at least one additive composition during application of the additive composition to the surface.

### FIELD OF THE INVENTION

The present invention generally relates to a cleansing device that includes a carrier to receive additive compositions, and more particularly relates to a cleansing device with a carrier that receives personal care additive compositions to be applied to a surface such as skin.

### BACKGROUND OF THE INVENTION

Humans have been making cleansing compositions since the times of ancient Babylon. In the almost five millennia since humans started making cleansing compositions, the compositions have taken a variety of forms, including both solid and liquid cleansing compositions. Such compositions are a regular part of many people's lives and in some cases are used on non-humans, such as animals. Compositions can be manufactured to perform a variety of functions. Specifically the different ingredients within a composition perform different functions. For example a composition may have ingredients that cleanse, remove odor, treat skin blemishes such as acne, moisturize the skin, prevent bacterial growth, and provide fragrance to the skin.

With such a wide variety of users having different skin types, the needs of distinct users may be different. For example, one individual may have dry skin and therefore may use a composition with an enhanced moisturizing property. A different individual that has acne or other skin blemishes would prefer a composition with an acne prevention or reduction property.

Accordingly, it is desirable to have a cleansing device that accommodates the different users and skin types that may use the cleansing device. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A cleansing device is described. The device includes a carrier having at least one cavity defined therein. The at least one cavity is to receive and maintain at least one additive composition. The at least one additive composition is to be applied to a surface.

The present application also describes a cleansing system. The system includes a carrier having at least one cavity defined therein. The at least one cavity is to receive and maintain at least one additive composition. The system also includes at least one additive composition disposed within a cavity of the carrier. The additive is to be applied to a skin surface.

The present application further describes a method for forming a carrier and additive compositions. The method includes forming a carrier having at least one cavity defined therein. A cavity to receive and maintain at least one additive personal care soap. The method also includes forming at least one additive personal care soap. The additive personal care soap is to be disposed within a cavity of the bar soap and is to be applied to a skin surface.

### BRIEF DESCRIPTION OF THE DRAWINGS

The present invention will hereinafter be described in conjunction with the following drawing figures, wherein like numerals denote like elements, and

FIG. 1 is a diagram of an example of a carrier of the cleansing device according to the principles described herein.

FIG. 2 is a diagram of an example of additive compositions according to the principles described herein.

FIG. 3 is a diagram of an example of a cleaning system with additive compositions disposed in a carrier according to the principles described herein.

FIG. 4 is a flowchart of a method for forming a carrier and an additive composition according to the principles described herein.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

As described above, compositions, and personal care compositions in particular, are commonly used by most individuals, and in some cases used on other animals. Such compositions are applied regularly; every day for example and in some cases multiple times per day. With varying needs based on such factors as skin type, environmental conditions, and budget, the needs of the users are at least as varied as the number of users themselves. For example, some users with dry skin, or that live in a dry climate may prefer a composition that has enhanced moisturizing characteristics than may a person living in a humid climate. As another example, some individuals may prefer a particular fragrance, or strength of fragrance where another may not. Addressing the various needs of the various users can become quite expensive to a producer and is a very complex task.

Accordingly, the principles described herein provide a customizable cleansing device. The device includes a base carrier that has cavities defined therein. Each of the cavities is to receive an additive composition, which additive composition may be configured to perform any variety of personal care functions. For example, an additive composition may include ingredients to 1) provide additional moisturization, 2) prevent or reduce acne, 3) exfoliate skin, 4) provide additional fragrance, and 5) mask or prevent odor. This is just a sample of the different functions, or ingredients that could be included in such an additive composition. A user may select which additive compositions to place in the base carrier such that the cleansing device may be selected, and customized, by a user depending on their particular needs or desires.

In this fashion, a user can form a customizable cleansing composition that is tailored to their particular needs. This may also be beneficial as a single composition, i.e., carrier with additives, may be used to deliver specific and customized personal care functions. This may be cheaper for a user as there is no need to buy multiple compositions, but a single composition with customizable additives. Such flexibility increases the ability of a manufacturer to meet the demands from the varied users with their various requirements. A composition that addresses the particular needs of a user is sure to create a satisfactory customer experience and could potentially result in increased profits for a producer of such a customized personal composition.

As used in the present specification and in the appended claims, the term “a number of” or similar language may include any positive number, including one to infinity; zero not being a number, but the absence of a number.

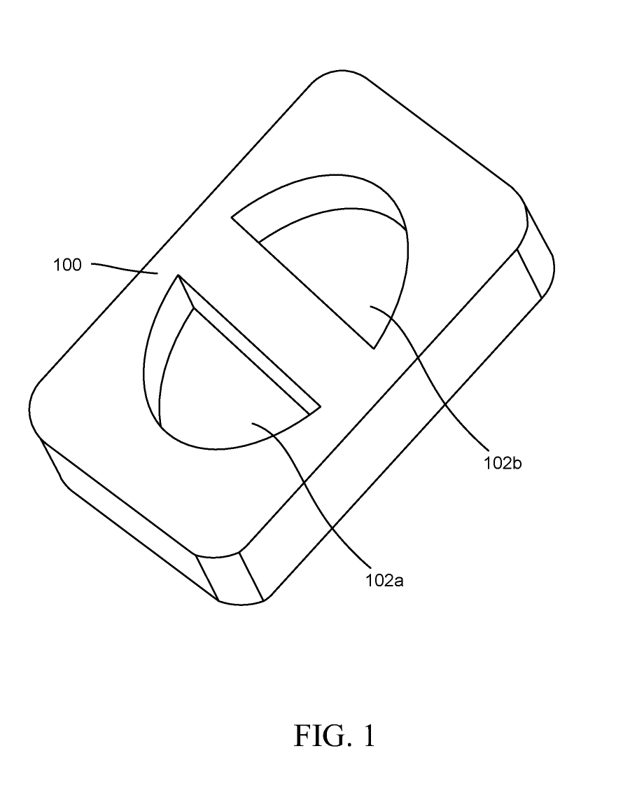

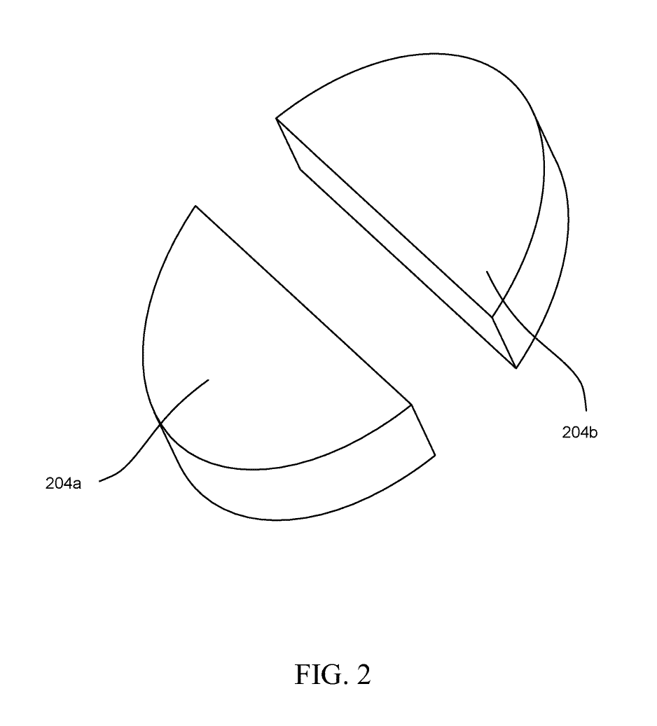

Turning now to the figures, FIG. 1 is a diagram of an example of a carrier (100) of the cleansing device according to the principles described herein. The carrier (100) houses the additive compositions that are described below in connection with FIG. 2. The carrier (100) is the vehicle by which a user applies the additive compositions to skin. For example, the user grasps the carrier (100) in their hand and rubs the carrier over the skin surface. Accordingly, the carrier (100) may be sized to fit in the hand of a user. During use, a friction force between the user's skin and the additive compositions dislodges particles from the compositions for deposition onto the skin. Additionally, when used in a wet environment such as a shower or a bath, the interaction with water also dislodges particles from the additive compositions, and in some cases the carrier (100), to be deposited on the skin surface.

The carrier (100) has at least one cavity (102) to receive the additive compositions, which additive compositions are to be applied to, and deposited on, a surface such as skin. As such, the cavities (102) are the same size and the same shape as the additive compositions that are to be deposited therein. While FIG. 1 depicts two cavities (102) of a particular size, shape, and orientation, the cavities (102) may be of any shape and size and placed anywhere on the carrier (100). For example, a first cavity (102a) may be placed on a front surface and a second cavity (102b) may be placed on an opposite surface. In this case, different additive compositions could be placed in either cavity (102) to provide different uses when different sides of the carrier (100) are exposed to skin.

Moreover, while FIG. 1 depicts a carrier (100) with two cavities (102a, 102b), the carrier (100) may have any number of cavities (102) to receive any number of additive compositions. For example, the carrier (100) may have multiple cavities (102) as indicated in FIG. 1, or the carrier (100) may have one cavity (102). In some examples, the cavities (102) may be through holes protruding through the carrier (100). Doing so may be beneficial as an inserted additive composition is exposed on at least two surfaces thus increasing the amount of composition available for deposition on a surface.

The carrier (100) may be formed of any material. For example, the carrier (100) may be a bar soap that includes some personal care ingredients. As used in the present specification and in the appended claims, the term ‘soap’ may refer to salts of fatty acids. During use an outer layer of the soap bar is removed and underneath layers are unaffected. In a soap form, the carrier (100) may include ingredients that cleanse the skin, or facilitate in the cleansing of skin. Such ingredients include a surfactant for cleansing or washing the skin, glycerol and a number of other additive ingredients.

The bar soap carrier (100) may be a semi-solid soap. A semi-solid soap is a soap that is not hard. In other words, the soap may be a soft solid. As a soft, semi-solid, or solid soap, the particles of the soap do not have sufficient inter-particulate cohesion to overcome the force of friction. In other words, as the soap carrier (100) is moved over a surface such as the skin, the friction force between the surface and the soap is greater than the cohesive bonds between the particles of the soap such that those particles are deposited on the surface and then carry out their intended function; whether that function be a cleansing, exfoliating, moisturizing, or odor-reducing function, among others. Still further, the interaction of the soap with water may also break down the bonds holding the individual soap particles together thus again resulting in the deposition of the soap carrier (100) particles on a skin surface.

In addition to these ingredients, the soap carrier (100) may include other ingredients to impart other functionality to the soap carrier (100). Such ingredients include a fragrance ingredient, exfoliating particles, and moisturizing ingredients among other personal care ingredients. In this example, the carrier (100), like the additive compositions, may carry out a personal care function when applied to skin. This additional skin maintenance is offered by a soap-like carrier (100).

In addition to the ingredients mentioned above, the soap carrier (100) may include other ingredients to improve the application aesthetics, contact aesthetics, or other characteristics of the soap carrier (100). For example, the soap carrier (100) may include a structurant. A structurant may be any compound or mixture configured to give shape, or structure to the soap carrier (100). Accordingly, the soap carrier (100) may include any compound or mixture that is configured to give shape, or structure to the soap carrier (100). Other examples of ingredients in the soap carrier (100) include an emollient, an anti-bacterial additive, a dye, an antioxidant, a skin conditioner, a pigment, and a preservative.

In other examples, the carrier (100) may be formed of a different material such as a silicon material, a rubber material, a foam material, and a sponge material. As described above a soap-type carrier (100) results in deposition of layers of the soap carrier (100) on the surface. However, with a silicon, rubber, plastic, foam, or sponge carrier (100), such deposition does not occur. Accordingly, the carrier (100) is reusable, even after the expiration of an inserted additive composition. As indicated in some of these examples, the carrier (100) may be formed of a porous material. A porous material enhances the formation of lather of the additive compositions thereby increasing their efficacy in cleaning the surface on which they are applied.

Using a carrier (100) that can facilitate different additive compositions is beneficial in that it allows a user to customize a cleansing device by selecting additive compositions that are relevant to that user. Such customization may reduce the cost of personal hygiene as a user can purchase a single base carrier and multiple additive compositions without having to purchase multiple full-size cleansing devices.

FIG. 2 is a diagram of an example of additive compositions (204) according to the principles described herein. As described above, additive compositions (204), which may be skin care compositions or personal care compositions are disposed in the cavities (FIG. 1, 102) of the carrier (FIG. 1, 100). The additive compositions (204) are the same shape and same size as the cavities (FIG. 1, 102) into which they are to be inserted. While FIG. 2 depicts an additive composition (204) of a particular size and shape, any size and shape of additive composition (204) can be used in line with the present application, so long as it fits the shape and size of the cavity (FIG. 1, 102), which cavity (FIG. 1, 102) also can be any size and shape.

The additive compositions (204) are used to provide various cleansing functions to a user. Accordingly, the additive compositions (204) may be of varying types, meaning the additive compositions (204) may include different compounds, such as personal care compounds that serve different functions. Examples of types of additive compositions (204) a moisturizer composition, a lotion composition, an anti-bacterial composition, an acne treatment composition such as salycilic acid, a sun block composition, an exfoliating composition, a deodorizing composition, a shaving cream composition, a body wash composition, an anti-oxidant composition, and a fragrance composition. The anti-bacterial composition may be triclosan, triclocarban, benzalkonium chloride, and benzethonium chloride among others. Each additive composition (204) may perform one or more of these functions. For example, a single additive composition (204) may be a combination of 1) have a moisturizing enhancing composition and 2) a fragrance composition.

As with the carrier (FIG. 1, 100), the additive compositions (204) may be soaps. The additive compositions (204) may be semi-solid compositions. Thus, in combination the additive compositions (204) may be semi-solid soaps. During use, particles of the soap to not have sufficient inter-particulate cohesion to overcome the force of friction. In other words, as the soap additive composition (204) is moved over a surface such as the skin, the friction force between the surface and the soap is greater than the cohesive bonds between the particles of the soap such that those particles are deposited on the surface and then carry out their intended function, whether that function be a cleansing, exfoliating, moisturizing, or odor-reducing function, among others. Still further the interaction of the soap with water may also break down the bonds holding the individual soap particles together thus again resulting in the deposition of the soap particles on a skin surface.

In addition to including ingredients carrying out the above described functions, the additive composition (204) may include other ingredients to improve the application aesthetics, contact aesthetics, or other characteristics of the additive composition (204). For example, the additive composition (204) may include a structurant. A structurant may be any compound or mixture configured to give shape, or structure to the additive composition (204). Accordingly, the additive composition (204) may include any compound or mixture that is configured to give shape, or structure to the additive composition (204). Other examples of ingredients in the additive composition (204) include an emollient, an anti-bacterial additive, a dye, an antioxidant, a skin conditioner, a pigment, and a preservative.

In some examples, different additive compositions (204) have different colors. For example, a first additive composition (204a) which may include an ingredient to prevent acne such as salycilic acid may be red color while a second additive composition (204b) which may include an extra moisturizer may be a blue color. Doing so may enable a user to more easily identify the purpose, or function, of a particular additive composition (204).

Producing different additive compositions (204) may be beneficial to a user by providing a number of different skin care compounds that a user can select and tailor to their preference. For example, a user with dry skin and that is prone to acne can select additives that provide an extra moisturizing ingredient and an acne prevention ingredient respectively. Such customization via additive compositions (204) placed in a carrier (FIG. 1, 100) are unique in the field.

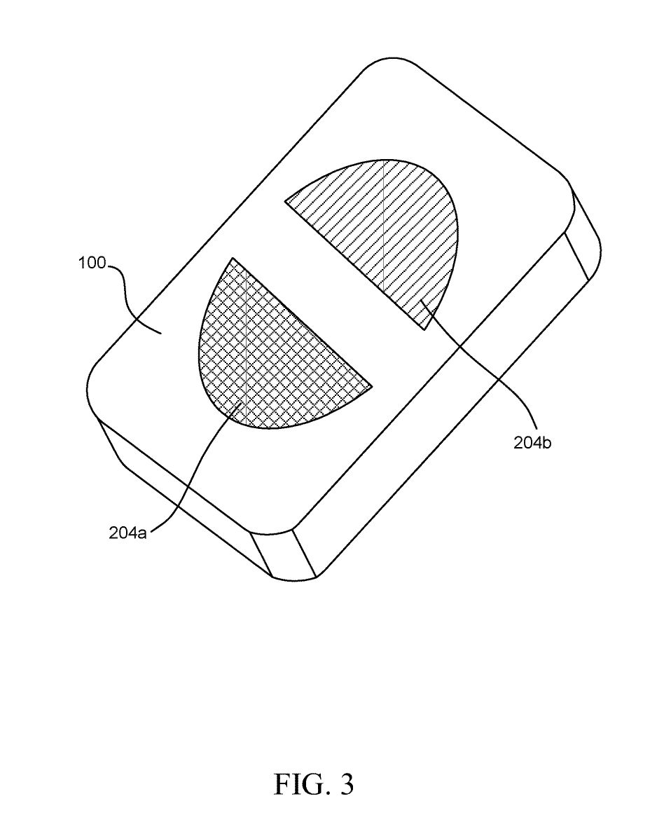

FIG. 3 is a diagram of an example of a cleaning system with additive compositions (204) disposed in a carrier (100) according to the principles described herein. As described above, the carrier (100) receives the additive compositions (204) such that the additive compositions (204) and the carrier (100) for a system. In other words, the cleaning system as described herein includes the carrier (100) having at least one cavity (FIG. 1, 102) and the additive compositions (204a, 204b) that are disposed therein.

Note that the hashing of the additive compositions (204a, 204b) is not meant to impart any material properties to the additive compositions (204a, 204b), but is merely intended to distinguish between the different additive compositions (204) and the carrier (100). The different hashing on the different additive compositions (204) indicates different types, or different purposes served by, the different additive compositions (204). That is the carrier (100) has multiple cavities (FIG. 1, 102) that not only house multiple additive compositions (204), but that house different types of additive compositions (204). For example, the first additive composition (204a) may be an additional fragrance composition while the second additive composition (204b) may be an odor-eliminating composition, or an antiperspirant composition. While FIG. 2, specifically depicts different kinds of additive compositions (204) in the different cavities (FIG. 1, 102), the different cavities (FIG. 1, 102) may be filled with different instances of the same composition (204). For example, a user could put two moisturizing additive compositions (204) in the carrier (100), one in either cavity (FIG. 1, 102) thus providing an even further enhanced moisturizing benefit.

The additive compositions (204) may be press fit into the carrier (100) so as to be retained in the carrier (100). Moreover, as water particles interact with the additive compositions (204) as well as with a non-synthetic carrier (100), the particles of the additive compositions (204) and non-synthetic carrier (100) may fuse to retain the additive compositions (204) in the carrier (100). Doing so may be beneficial for improving the efficacy of the cleansing system. For example, if the additive composition (204) falls out of the carrier (100), a user may become frustrated by continually having to re-position the additive composition (204) within the carrier (100). When joined as depicted in FIG. 3, the system is ready for use.

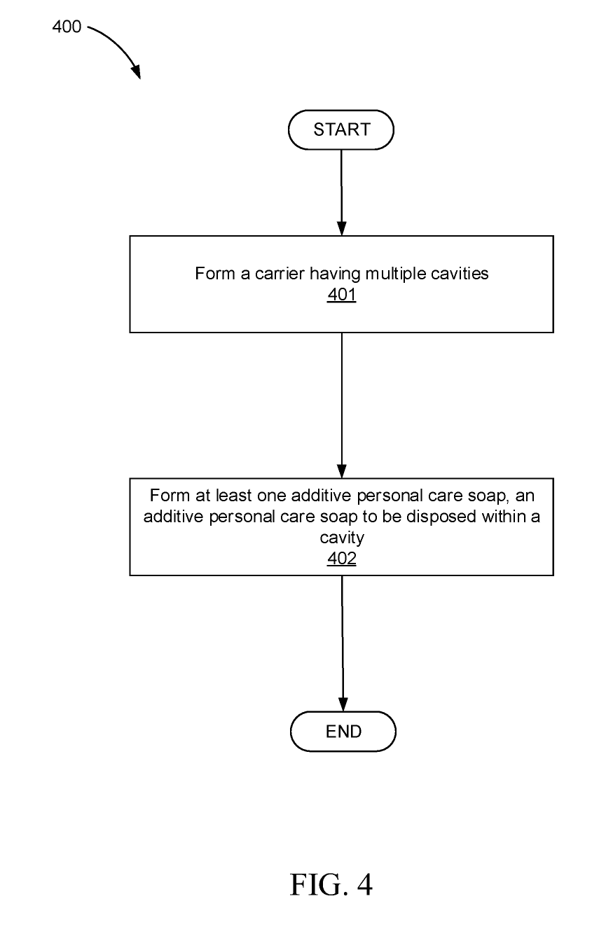

FIG. 4 is a flowchart of a method (400) for forming a carrier (FIG. 1, 100) and an additive composition (FIG. 2, 204) according to the principles described herein. According to the method (400), a carrier (FIG. 1, 100) is formed (block 401). The carrier (FIG. 1, 100) has multiple cavities which are each to receive an additive personal care soap, the personal care soap being an example of an additive composition. Forming (block 401) the carrier (FIG. 1, 100) depends on the type of carrier (FIG. 1, 100) used. For example, if the carrier (FIG. 1, 100) is a silicon, plastic, or rubber material, the carrier (FIG. 1, 100) may be formed by a molding process. In this example, the cavities (FIG. 1, 102) that are defined in the carrier (FIG. 1, 100) may be part of the mold and formed at the same time. In a similar, example when the carrier (FIG. 1, 100) is formed out of a soap material, the soap material may be positioned with in a mold (FIG. 1,100) and formed into the desired shape, such as a solid bar, or a stick. As another example, the carrier (FIG. 1, 100) may be formed by compressing particulate matter into the desired shape. As described above, in some examples, the cavities (FIG. 1, 102) are through holes. In this example, the method (400) includes forming cavities (FIG. 1, 102) in the carrier (FIG. 1, 100) by punching holes through the carrier (FIG. 1, 100).

The method (400) also includes forming (block 402) additive personal care soaps, which are examples of additive compositions. The additive personal care soaps are formed in a similar fashion to the carrier (FIG. 1, 100) that is formed of a soap-like material. That is the additive personal care soaps may be formed by compression or molding. As the additive personal care soaps are disposed within the cavities (FIG. 1, 102) of the carrier (FIG. 1, 100), forming (block 402) the additive personal care soaps includes forming them to be the same size and shape as the cavities (FIG. 1, 102) of the carrier (FIG. 1, 100). As described above, a system using a base carrier (FIG. 1, 100) with interchangeable inserts is beneficial in that a user may tailor the cleansing device to their particular wants or needs.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1. A cleansing device comprising:
a carrier, which is a bar soap, having at least one through hole cavity defined therein; and
at least one additive composition being sized and shaped to be received and maintained in at least one cavity.

2. (canceled)

3. The device of claim 2, wherein the bar soap comprises skin care ingredients.

4. The device of claim 1, wherein the carrier comprises multiple cavities.

5. The device of claim 1, wherein the carrier is a porous material.

6. The device of claim 1, wherein the carrier is made from at least one of a silicon material, a rubber material, a foam material, and a sponge material. (Currently Amended) A cleansing system comprising:
a carrier having at least one through hole cavity defined therein, wherein the at least one cavity is to receive and maintain at least one additive composition; and
at least one additive composition disposed within a through hole cavity of the carrier, wherein the additive composition is the same shape and size as the at least one cavity and is to be applied to a skin surface.

8. The cleansing system of claim 7, wherein the at least one additive composition is a semi-solid composition.

9. The cleansing system of claim 7, wherein the at least one additive composition is a soap composition.

10. The cleansing system of claim 7, wherein the carrier is a solid bar soap.

11. The cleansing system of claim 7, wherein the at least one additive composition includes at least one personal care compound.

12. The cleansing system of claim 11, wherein the at least one personal care compound comprises a moisturizer, a lotion, an antibacterial compound, an acne treatment, a sun block compound, an exfoliating compound, a deodorizing compound, a shaving cream compound, a body wash compound, an anti-oxidant compound, and a fragrance compound.

13. The cleansing system of claim 12, wherein the antibacterial compound comprises at least one of triclosan, triclocarban, benzalkonium chloride, and benzethonium chloride.

14. (canceled)

15. The cleansing system of claim 7, comprising multiple and distinct types of additive composition.

16. The cleansing system of claim 7, wherein different additive composition have different colors.

17. A method for making a carrier and at least one additive personal care soap comprising:
forming a carrier having multiple cavities defined therein, wherein a cavity is to receive and maintain the additive personal care soap; and
forming the additive personal care soap, wherein the additive personal care soap is disposed within a cavity of the bar soap and is to be applied to a skin surface.

18. The method of claim 17, wherein forming the carrier comprises forming the carrier out of a soap material.

19. The method of claim 17, further comprising forming the cavities in the carrier, wherein the cavities are through holes protruding through the carrier.

20. The method of claim 17, wherein the additive personal care soap is formed to be the same size and shape as a cavity.
